(19) United States
(12) Patent Application Publication
(10) Pub. No.: US 2026/0048079 A1
(43) Pub. Date: Feb. 19, 2026

(54) DIETARY SUPPLEMENTS COMPRISING SILICA PARTICLES

(71) Applicant: Sigrid Therapeutics AB, Stockholm (SE)

(72) Inventors: Ghislaine Monique Nicole Robert-Nicoud, Stockholm (SE); Eric Johnston, Bromma (SE); Tore Bengtsson, Vaxholm (SE)

(21) Appl. No.: 19/105,140

(22) PCT Filed: Aug. 23, 2023
(86) PCT No.: PCT/EP2023/073130

§ 371 Date: Feb. 20, 2025

(30) Foreign Application Priority Data
Aug. 23, 2022 (GB) 2212239.4

(51) Int. Cl. A61K 33/00; A61K 47/36; A61K 47/38

(52) U.S. Cl. CPC A61K 33/00; A61K 47/36; A61K 47/38

(57) ABSTRACT

There is provided herein an oral dietary supplement comprising: (a) water. (b) silica particles, and (c) a thickening agent, wherein the thickening agent is Xanthan gum; and formulations thereof.

### FIELD OF THE INVENTION

The present invention relates to novel formulations and uses of such formulations, such as their use as food supplements.

In particular, the present invention relates to novel aqueous formulations, comprising silica particles and a thickening agent, for oral administration, and to the use of such formulations as orally consumable dietary supplements.

### BACKGROUND OF THE INVENTION

The listing or discussion of any prior-published document in this specification should not necessarily be taken as an acknowledgement that the document is part of the state of the art or is common general knowledge.

The use of silica as a food supplement is associated with numerous health benefits. Depending on the type of silica administered, these benefits may include promoting healthy hair, skin and nails, improving joint, bone and cartilage health, promoting detoxification of the body, boosting collagen production, aiding digestion and improving heart health (see, for example, Martin, K. R., J Nutr Health Aging, 11 (2), 94-7 (2007)).

It has also been shown that silica particles with specific porosity can lower adipose tissue in animal model systems (see: WO 2014/072363 and Kupferschmidt, N. et al., Nanomedicine, 9 (9), 1353-1362 (2014)) and may also reduce levels of HbAlc, which is a biomarker the levels of which correspond to long-term plasma glucose levels (see Baek, J. et al., Nanomedicine, 17:1, 9-22 (2022)).

Ideally, silica is administered in the form of silica particles, which may be orally consumed either with or without food. However, the use of silica particles as an oral food supplement poses significant challenges. In particular, the amount of silica material required in order to achieve a therapeutic effect is often relatively large, typically in the region of several grams, which renders the presentation of such material in the form of tablets or capsules inconvenient, as limitations on the size and shape of individual tablets and capsules suitable for oral administration would result in a need for the consumption of multiple dosage units.

In order to address this problem, silica material may be presented in the form of an orally consumable liquid. However, such liquid formulations suffer from instability, in that the silica material may separate from the formulation such that it becomes unevenly distributed, and a poor taste profile, such as having a gritty texture and inducing feelings of dry mouth following consumption.

There exists, therefore, a need for oral formulations of silica material that are stable and have a pleasant texture, such that they are more readily consumed.

### DETAILED DESCRIPTION OF THE INVENTION

It has unexpectedly been found that improved oral formulations of silica material may be prepared through the addition of a specific thickening agent. Such formulations may be of use as dietary supplements.

Novel formulations

In a first aspect of the invention, there is provided an oral dietary supplement comprising:
(a) water;
(b) silica particles; and
(c) a thickening agent, wherein the thickening agent is Xanthan gum.

Unless indicated otherwise, all technical and scientific terms used herein will have their common meaning as understood by one of ordinary skill in the art to which this invention pertains.

For the avoidance of doubt, the oral dietary supplement may also be referred to as an oral formulation, and vice versa.

Dietary supplement formulations as defined in the first aspect of the invention (including all embodiments and features thereof) may also be referred to as “the formulation(s) of the invention (or of the first aspect of the invention),” or the like. Such formulations may also be referred to as compositions, which terms may be used interchangeably. Similarly, the silica particles referred to in the first aspect of the invention (including all embodiments and features thereof) may also be referred to as “the silica (or silica material or silica particles) of the invention (or of the first aspect of the invention),” or the like.

When used herein in relation to a specific value (such as an amount), the term “about” (or similar terms, such as “approximately”) will be understood as indicating that such values may vary by up to 10% (particularly, up to 5%, such as up to 4%, 3%, 2% or 1%) of the value defined. It is contemplated that, at each instance, such terms may be replaced with the notation “±10%”, or the like (or by indicating a variance of a specific amount calculated based on the relevant value). In relation to percentage amounts referred to herein, the term “about” (or similar terms, such as “approximately”) will be understood as indicating that such values may vary by up to 10% (particularly, up to 5%, such as up to 4%, 3%, 2% or 1%) of the percentage value defined. It is also contemplated that, at each instance, the term about may be deleted.

For the avoidance of doubt, references to formulations comprising certain components will include the possibility that such formulations may consist of (or consist substantially of) those components.

For the avoidance of doubt, the skilled person will understand that where percentages of a certain component are defined as belonging to different (i.e. non-overlapping) groups, the sum of those percentages cannot exceed 100%. Thus, where the maximum values of one or more non-overlapping groups of components would exceed 100%, the skilled person will understand that one or more of those components must be present in an amount that is less than the maximum value. Similarly, where it is possible for other components to be present in a formulation, there is no requirement for the sum of the specified components to equal 100%.

The skilled person will understand that references to an oral formulation will refer to a formulation for (e.g. suitable for) oral consumption, such as oral consumption by a human or animal (e.g. a mammal, such as a household pet or recreational animal, such as a cat, dog, horse, rabbit, or the like, or livestock, such as a cattle, pigs, sheep, goats, chickens, turkeys, or the like). In certain instances, references to consumption by such human or animal subject (which, in relation to methods of medical treatment, may also be referred to as patients), will refer in particular to consumption by adult (e.g. fully grown) subjects.

For the avoidance of doubt, the skilled person will understand that references to an oral formulation include an orally ingestible (i.e. suitable for being ingested) product, such as a medical device.

In particular, references to an oral formulation will refer to a formulation for (e.g. suitable for) oral consumption by a human, such as an adult human (e.g. a human of at least 18 years of age).

The skilled person will understand that references herein to oral consumption will refer to the formulation being swallowed, either in one action or several, by the subject. As such, the skilled person will understand that the formulation of the invention may be in the form of a liquid, such that the formulation may be easily swallowed in the manner of a drink.

Thus, in particular embodiments, the formulation is a liquid (in which case it may be referred to as a liquid formulation).

The skilled person will understand that the term liquid will typically refer to a substance that flows freely but is of constant volume, typically having a consistency similar to that of water or a liquid oil.

The skilled person will understand that the amount of each component present in the formulation may be adjusted in order to achieve the required viscosity and flowability.

In particular embodiments, the formulation may be provided in a form having a viscosity classified as low to medium, such as having a viscosity and/or flowability similar to that of a milkshake or similar beverage/food item.

As described herein, the formulation of the invention comprises water. In particular, the formulation will typically comprise water as a significant component, such as being the major (i.e. largest) component by weight. The formulation may therefore be described as being an aqueous (or substantially aqueous) formulation.

The skilled person will understand that the amount of water included in the formulation may be selected in order to achieve the desired properties of the formulation, such as the desired viscosity and/or flowability.

In particular embodiments, the formulation comprises:
at least about 70.0% (such as at least about 71.0, 72.0, 73.0, 74.0, 75.0, 76.0, 77.0, 78.0, 79.0%) by weight (wt %) of water; and/or (e.g. and)
at least about 80.0 wt % (such as at least about 81.0, 82.0, 83.0, 84.0 or 85.0 wt %) water.

In particular embodiments, the formulation comprises up to about 95.0 wt % (such as up to about 94.0, 93.0, 92.0 or 91.0 wt %) water.

In more particular embodiments, the formulation comprises up to about 90.0 wt % (such as up to about 89.0, 88.0, 87.0, 86.0 or 85.0 wt %) water.

For example, in particular embodiments, the formulation comprises from about 70.0 to about 95.0 wt % water.

In more particular embodiments, the formulation comprises from about 75.0 to about 95.0 wt % water.

In more particular embodiments, the formulation comprises from about 80.0 to about 95.0 wt % water.

In more particular embodiments, the formulation comprises from about 75.0 to about 90.0 wt % water.

In more particular embodiments, the formulation comprises from about 80.0 to about 90.0 wt % water.

In more particular embodiments, the formulation comprises from about 82.0 to about 88.0 wt % water.

In more particular embodiments, the formulation comprises from about 83.0 to about 87.0 wt % water.

In more particular embodiments, the formulation comprises from about 84.0 to about 86.0 wt % water.

In a certain embodiment, the formulation comprises from about 84.0 to about 91.0 wt % water.

In a certain embodiment, the formulation comprises from about 84.0 to about 90.0 wt % water.

In a particular embodiment, the formulation comprises about 85 to about 86 wt % water, such as about 85.6 wt % water.

In a particular embodiment, the formulation comprises about 89 to about 90% wt % water, such as about 89.0 to about 90.0% wt % water.

In certain embodiments, it may be understood that, where the amounts of other ingredients are specified, the remainder of the composition (e.g. by weight) is water.

As described herein, the formulation of the invention comprises silica particles. The skilled person will understand that the amount of such silica particles in the formulation may be selected in order to provide an appropriate loading such that administration of a suitable amount of the formulation delivers the desired amount of silica material (e.g. the dose required in order to achieve the desired health benefits).

In particular embodiments, the formulation comprises:

at least about 5.0 wt % (such as at least about 6.0, 7.0, 8.0 or 9.0 wt %) of the silica particles; and/or (e.g. and) up to about 15.0 wt % (such as at least about 14.0, 13.0, 12.0 or 11.0 wt %) of the silica particles.

In particular embodiments, the formulation comprises from about 5.0 to about 15.0 wt % of the silica particles.

In more particular embodiments, the formulation comprises from about 6.0 to about 14.0 wt % of the silica particles.

In more particular embodiments, the formulation comprises from about 7.0 to about 13.0 wt % of the silica particles.

In more particular embodiments, the formulation comprises from about 8.0 to about 12.0 wt % of the silica particles.

In more particular embodiments, the formulation comprises from about 9.0 to about 11.0 wt % of the silica particles.

In more particular embodiments, the formulation comprises from about 9.0 to about 10.5 wt % of the silica particles.

In particular embodiments, the formulation comprises from about 9.0 to about 10.0 wt % of the silica particles, such as about 10.0 wt % or about 9.4 wt % of the silica particles.

As described herein, the formulation of the invention comprises a thickening agent, wherein the thickening agent is Xanthan gum.

As described herein, it has been found that the use of this thickening agent allows for the preparation of formulations that provide improved properties in terms of stability, texture and palatability.

The skilled person will be able to select a suitable amount of the thickening agent in order to achieve required properties of the formulation, such as the viscosity and flowability thereof.

As described herein, it has been found that certain amounts (wt %) of the thickening agent xanthan gum unexpectedly provide increased long-term stability of the formulation.

As such, in particular embodiments, the formulation comprises from about 0.4 wt % to about 2.0 wt % of the thickening agent.

In more particular embodiments, the formulation comprises from about 0.4 wt % to about 1.9 wt % of the thickening agent.

In more particular embodiments, the formulation comprises from about 0.4 wt % to about 1.8 wt % of the thickening agent.

In yet more particular embodiments, the formulation comprises from about 0.4 wt % to about 1.7 wt % of the thickening agent.

In yet more particular embodiments, the formulation comprises from about 0.4 wt % to about 1.6 wt % of the thickening agent.

In yet more particular embodiments, the formulation comprises from about 0.4 wt % to about 1.5 wt % of the thickening agent.

In yet more particular embodiments, the formulation comprises from about 0.4 wt % to about 1.4 wt % of the thickening agent.

In yet more particular embodiments, the formulation comprises from about 0.4 wt % to about 1.3 wt % of the thickening agent.

In yet more particular embodiments, the formulation comprises from about 0.4 wt % to about 1.2 wt % of the thickening agent.

In yet more particular embodiments, the formulation comprises from about 0.5 wt % to about 1.2 wt % of the thickening agent.

In yet more particular embodiments, the formulation comprises from about 0.5 wt % to about 1.1 wt % of the thickening agent.

In yet more particular embodiments, the formulation comprises from about 0.6 wt % to about 1.1 wt % of the thickening agent.

In yet more particular embodiments, the formulation comprises from about 0.5 wt % to about 1.0 wt % of the thickening agent.

In yet more particular embodiments, the formulation comprises from about 0.6 wt % to about 1.0 wt % of the thickening agent (e.g. about 0.6 or about 1.0 wt % of the thickening agent).

In certain embodiments, the formulation comprises from about 0.4 wt % to about 0.8 wt % of the thickening agent.

In certain embodiments, the formulation comprises from about 0.5 wt % to about 0.7 wt % of the thickening agent.

For example, in particular embodiments, the formulation comprises about 0.6 wt % of the thickening agent.

The skilled person will understand that the formulation of the invention may comprise further components in order to adjust and/or preserve properties such as the freshness/food safety (i.e. being free from contaminants, such as bacterial and mould growth), taste, smell and/or colour of the formulation.

Thus in particular embodiments, the formulation further comprises:
(d) optionally, one or more preservative, sweetener, flavouring, fragrance and/or colorant.

In particular embodiments, the amount of optional component (d) present in the formulation is up to about 10.0 wt % (such as up to about 9.0, 8.0, 7.0 or 6.0 wt %) of the formulation.

In more particular embodiments, the amount of optional component (d) present in the formulation is up to about 5.0 wt % (such as up to about 4.9, 4.8, 4.7, 4.6, 4.5 or 4.4 wt %) of the formulation.

For example, the formulation may further comprise one or more preservative, such as those food preservative agents suitable for human consumption as known to those skilled in the art.

For example, suitable preservatives will include lactic acid, benzoic acid and pharmaceutically acceptable salts thereof (e.g. sodium benzoate), sorbic acid and pharmaceutically acceptable salts thereof (e.g. potassium sorbate) and citric acid, including mixtures thereof.

Particular preservatives that may be mentioned include citric acid and potassium sorbate, including mixtures thereof.

For the avoidance of doubt, preservatives, such as those described herein, may also be referred to as acidity modifiers.

In a particular embodiment, the formulation further comprises one or more preservatives.

For example, in a particular embodiment, the formulation comprises the preservatives citric acid (e.g. in an amount of about 0.1 to about 0.3 wt % of the formulation, such as about 0.2 wt % of the formulation, e.g. about 0.22 wt % of the formulation) and potassium sorbate (e.g. in an amount of about 0.05 to about 0.15 wt % of the formulation, such as about 0.1 wt % of the formulation, e.g. about 0.09 wt % of the formulation).

The formulation may also comprise one or more flavouring agent and/or sweetener, such as those flavouring agents and sweeteners suitable for human consumption as known to those skilled in the art.

For example, suitable sweeteners may include sugars (such as sucrose, tagatose and allulose), sugar alcohols (such as erythritol, xylitol, sorbitol and mannitol), artificial sweeteners (such as sucralose, saccharin, acesulfame-K and aspartame) and plant extracts (such as steviol glycosides and monk fruit extract). Particular sweeteners that may be mentioned include non-caloric sweeteners, particularly sugar alcohols (such as erythritol) and plant extracts (such as steviol glycosides).

In a particular embodiment, the formulation comprises one or more sweetener.

The skilled person will be able to select a suitable sweetener and amount thereof in order to achieve the required taste profile of the formulation.

In particular embodiments, the sweetener is present in an amount of about 0.01 to about 10.0 wt % of the formulation.

In more particular embodiments, the sweetener is present in an amount of about 0.05 to about 10.0 wt % of the formulation.

In yet more particular embodiments, the sweetener is present in an amount of about 0.1 to about 10.0 wt % of the formulation.

In yet more particular embodiments, the sweetener is present in an amount of about 0.5 to about 10.0 wt % of the formulation.

In yet more particular embodiments, the sweetener is present in an amount of about 1.0 to about 10.0 wt % of the formulation.

In yet more particular embodiments, the sweetener is present in an amount of about 2.0 to about 9.0 wt % of the formulation.

In yet more particular embodiments, the sweetener is present in an amount of about 2.0 to about 8.0 wt % of the formulation.

In yet more particular embodiments, the sweetener is present in an amount of about 2.0 to about 7.0 wt % of the formulation.

In yet more particular embodiments, the sweetener is present in an amount of about 2.0 to about 6.0 wt % of the formulation.

In yet more particular embodiments, the sweetener is present in an amount of about 3.0 to about 5.0 wt % of the formulation.

In yet more particular embodiments, the sweetener is present in an amount of about 3.0 to about 4.0 wt % of the formulation.

In yet more particular embodiments, the sweetener is present in an amount of about 3.5 to about 3.9 wt % of the formulation.

In yet more particular embodiments, the sweetener is present in an amount of about 3.6 to about 3.9 wt % of the formulation.

In particular embodiments, the sweetener is present in an amount of about 4 wt % of the formulation, such as about 3.8 wt % of the formulation.

In a more particular embodiment, the sweetener is erythritol or a mixture of erythritol and steviol glycoside.

In particular such embodiments (i.e. where the sweetener is erythritol and steviol glycoside), the sweetener is present in an amount of about 4 wt % of the formulation, such as about 3.8 wt % of the formulation, e.g, wherein the erythritol is present in an amount of about 3.7 to about 3.8 wt % of the formulation, such as about 3.75 wt % of the formulation, and the steviol glycoside is present in an amount of about 0.01 wt % of the formulation).

As described herein, the formulation may additionally comprise a flavouring (for example, vanilla and/or fruit flavourings), which may derive from an extract and/or artificial agents.

Such flavourings may be present in an amount that is about 0.01 to about 0.50 wt % of the formulation, e.g. about 0.01 to about 0.30 wt % of the formulation, such as about 0.25 wt % of the formulation.

In a particular embodiment, the formulation does not contain (or is substantially free of) additional active agents (i.e. active agents other than the silica material of the invention).

For the avoidance of doubt, the formulation may comprise further additional components, which may be selected in order to enhance and/or supplement the properties of the formulation.

For example, the skilled person will understand that other such additional components, such as colorants and fragrances, may be selected in order to provide a formulation that is appealing to the subject. Where present, such components may be present in an amount that is essentially negligible when compared to the other components.

In particular embodiments, the amounts of components (i.e. components (a) to (c), or (a) to (d), as required) as specified herein are equal to 100% (in which embodiments, it may be said that the formulation consists of, or consists essentially of, components as specified herein).

In other embodiments, other components may be added to the formulations of the invention, in amounts that may be additional to the components and relative amounts thereof as specified above, without altering the properties of the formulation. In such embodiments, the formulation comprises components (i.e. components (a) to (c), or (a) to (d), as required) as specified herein (with relative amounts thereof equal to 100%), optionally comprising further components.

The skilled person will understand that formulations of the invention will contain each component as a mixture thereof. In particular, such mixtures will contain each component such that it is evenly distributed throughout the formulation, which formulations may be described as being homogenous (or substantially homogenous).

As described herein, formulations of the invention will be stable, which may indicate that the composition of the formulation and the distribution of such components therein will not change over an extended period of time, such as a period of at least 42 days (such as at least 1 year (365 days)).

In particular embodiments, the formulation will be stable for at least 42 days (such as at least 1 year (365 days)), such as wherein the formulation will remain homogenous (or substantially homogenous) upon storage for a period of at least 42 days (such as at least 1 year (365 days); e.g. at room temperature and pressure, when shielded from humidity and sunlight).

Silica particles

The skilled person will understand that the silica particles as provided in the formulations of the first aspect of the invention may be referred to as a plurality thereof, which plurality may be referred to as a silica material.

For the avoidance of doubt, such silica particles may be porous, such as silica particles having pores in the mesoporous range (which may be referred to as mesoporous silica particles). Porous silica materials that may be of use include those described in Baek, J. et al., Nanomedicine, 17:1, 9-22 (2022), the contents of which are hereby incorporated herein by reference.

The skilled person will also understand that the silica particles may have a particular surface area, which may be affected by properties such as the porosity thereof.

The skilled person will also understand that the silica particles may be provided in a variety of shapes, such as being spherical or rod-shaped, and may vary in relation to their particle size.

The skilled person will understand that the term mean particle size, as used herein, will refer to the mean diameter of the particles at the greatest point thereof (e.g. in the case of rod-shaped particles, the length thereof; or in the case spherical particles, the diameter thereof.), which may be measured using techniques well-known to those skilled in the art, for example using electron microscopy techniques (such as by Scanning Electron Microscopy (SEM) or Transmission Electron Microscopy (TEM) technique known to those skilled in the art). In a particular embodiment, particle size is determined using electron microscopy (e.g. using SEM) or by laser diffraction.

Where the particles are spherical, the size of particles (as measured by SEM) may be defined by reference to the diameter thereof, such as having a mean particle size of:
from about 0.1 to about 20.0 μm;
from about 0.1 to about 15.0 μm;
from about 0.1 to about 10.0 μm;
from about 0.5 to about 10.0 μm;
from about 0.5 to about 5.0 μm;
from about 0.5 to about 4.5 μm;
from about 1.0 to about 10.0 μm;
from about 1.0 to about 5.0 μm;
from about 1.0 to about 4.0 μm;
from about 1.0 to about 4.0 μm;
from about 2.0 to about 4.0 μm; or
from about 3.0 to about 4.0 μm.

Where the particles are rod-shaped, the size of particles (as measured by SEM) may be defined (or also defined) by reference to the width thereof, such as having a mean width of:
from about 0.05 to about 0.6 μm;
from about 0.1 to about 0.6 μm;
from about 0.1 to about 0.4 μm; or
from about 0.2 to about 0.4 μm.

Where the particles are spherical, the size of particles (as measured by laser diffraction) may be defined by reference to the diameter thereof, such as having a mean particle size of:
from about 1.5 to about 20.0 μm;
from about 1.5 to about 15.0 μm;
from about 1.5 to about 10.0 μm;
from about 2.0 to about 10.0 μm;
from about 2.0 to about 7.5 μm;
from about 2.0 to about 5.0 μm;
from about 2.0 to about 4.5 μm;
from about 2.5 to about 4.5 μm;
from about 3.0 to about 4.5 μm;
from about 3.5 to about 4.5 μm; or
from about 3.5 to about 4.5 μm.

Where the particles are rod-shaped, the size of particles (as measured by laser diffraction) may be defined (or also defined) by reference to the width thereof, such as having a mean width of:
from about 1.5 to about 20.0 μm;
from about 2.0 to about 10.0 μm;
from about 2.0 to about 5.0 μm; or
from about 3.5 to about 4.5 μm.

The skilled person will understand that silica materials of the type described in the present invention are typically non-crystalline. Thus, in certain embodiments, the silica particle may be described as a substantially non-crystalline silica particle (and materials formed from a plurality of such particles may be described in the same manner). As such, the silica particles may be described as non-crystalline silica particles.

In alternative embodiments, the silica material present in particles as described in the first aspect of the invention may be described as being amorphous. In such embodiments, it will be understood that the term amorphous will indicate that the structure of the silica material (excluding the pores present therein) has no substantial order, such as the order which may be present in a crystalline substance (i.e. the silica particles, or silica material, may be referred to as non-crystalline).

Use as a dietary supplement

As described herein, the formulation of the invention may be useful as a dietary supplement, and may therefore be provided in a form suitable for such uses. As such, the formulation of the invention may be described as a dietary supplement formulation (e.g. an oral dietary supplement formulation).

In a second aspect of the invention, there is provided a formulation as defined in the first aspect of the invention (including all embodiments and features thereof) for use as a dietary supplement.

For the avoidance of doubt, formulations referred to as a dietary supplement formulation may also be suitable for use as food supplement, and vice versa. Thus, the formulations of the invention may be referred to as a food supplement, or the like.

The skilled person will understand that formulations of the invention may be taken (i.e. administered) at the same time as, or as part of, food (e.g. a meal) or a drink.

The skilled person will understand that references herein to administration with food (or, similarly, to administration with a meal) or drink may refer to administration at the same time as the food or drink is consumed, or shortly before or after the consumption of food or drink (e.g. up to 2 hours, such as up to 1 hour or, particularly, up to 30 minutes, before or after the consumption of food).

The skilled person will also understand that the formulation of the invention, when used as a dietary supplement, may induce biological effects as described herein. As such, a dietary supplement formulation as described herein may also be referred to as an active dietary supplement formulation.

The skilled person will understand that references to the formulation when used as a dietary supplement refers to the formulation being orally consumed (i.e. is consumed orally) by a human or animal (e.g. a mammal, such as a household pet or recreational animal, such as a cat, dog, horse, rabbit, or the like, or livestock, such as a cattle, pigs, sheep, goats, chickens, turkeys, or the like).

Packaging and manufacture

The skilled person will appreciate that the formulation of the invention may be packaged (e.g. for distribution and sale) in any suitable manner for a dietary or food supplement, as appropriate for the intended use.

For example, the formulation of the invention may be packaged in single portion containers, such as containers (e.g. single use bottles or pouches, or stick packs) from which the formulation may be consumed directly.

The formulations of the invention may be prepared using techniques known to those skilled in the art, such as by mixing of the components of the composition (in one process or sequentially in sub-sets thereof) to achieve a substantially homogenous mixture thereof.

Thus, in a further aspect of the invention, there is provided a process of preparing a formulation according to the first aspect of the invention (including all embodiments and features thereof), comprising the step of bringing the components of the formulation together to form a mixture thereof and homogenising said mixture.

Without wishing to be bound by theory, it is believed that formulations of the invention allow for silica particles to be provided in a form that is stable and easily consumed by a subject, without the unpleasant mouth feel (i.e. grittiness and dry after-taste) associated with other formulations, thus increasing the palatability of the formulation.

### Examples

The present invention will be further described by reference to the following examples, which are not intended to limit the scope of the invention.

### Materials

Silica particles were mesoporous spherical silica particles (MSP) with an average particle size of 4 μm measured by laser diffraction and 1.5 μm measured by Scanning Electron Microscopy (SEM).

Particle size measured by Laser Diffraction

A Mastersizer 3000 equipped with a Hydro LV dispersion unit (Malvern Panalytical, Malvern, UK) was used to measure the size of silica particles dispersed in water. The silica powder was added to the dispersion unit while stirring (3500 rpm) until a minimum of 5% laser obscuration was reached. Stirring was continued for 2 minutes, followed by 3 minutes of sonication, prior to particle size measurements. The average particle size was reported as the median particle size calculated from the volume particle size distribution (n=3).

Particle size measured by SEM

Scanning electron microscopy using a JSM-7401F (JEOL Ltd., Tokyo, Japan) was used to characterize the particle size and morphology from SEM micrographs. The mean particle size (diameter of spherical particles) were analysed from >100 particles using Image] (Fiji; see Schindelin J, Arganda-Carreras I, Frise E et al., Fiji: an open-source platform for biological-image analysis., Nat. Methods, 9 (7), 676-682 (2012)).

The following ingredients were purchased from Sigma-Aldrich: potassium sorbate (1.05119.1000), Karaya gum (G0503), 2-hydroxyethyl cellulose (434981), Sodium carboxymethylcellulose (419303) and guar gum (G4129). Xanthan gum was obtained from CP Kelco (Keltrol® RD). Tragacanth gum was purchased from Rainbow Dust Colours. Colloidal crystalline cellulose was obtained from JRS Pharma (Vivapur® MCG 900XF). Flavourings were obtained from Einar Willumsen: vanilla extract (22219), blueberry flavour (35144) and peach flavour (12101). Erythritol was purchased from Amazon (ASIN B01MFCXDZA). Steviol glycosides were obtained from Tate & Lyle (Intesse™ Stevia 2.0).

### Example 1: Preparation of Formulations

Preparation of stock solutions

Solution 1: Potassium sorbate was dissolved in water. Citric acid was then added to the same solution and stirred until dissolved. The concentration of potassium sorbate was 0.125 wt % and the concentration of citric acid was 0.250 wt %

Solution 2: Steviol glycosides (0.4 g) were dissolved in 20 g water.

Solution 3: Blueberry flavour (0.6 g) was diluted in 20 g water.

Solution 4: Peach flavour (0.6 g) was diluted in 20 g water.

### Formula 1-15

The thickening agent (concentration according to Table 1; also referred to as a thickener) was slowly added to 40.15 g of Solution 1 and water (amount adjusted to have a total weight of 50 g) while homogenizing. The homogenization speed was slowly increased from low to medium speed as the formulation thickened. Following addition of the thickener, the formulations were homogenized at medium speed for another 3 min. The thickener appeared fully dispersed (lumps no longer visible). Finally, 5 g of MSP were added to the formulation and homogenized at medium speed for 3 min.

### Formula 16

Potassium sorbate (0.2 g) was dissolved in water (163.28 g). Vanilla extract (0.32 g), Solution 2 (1.0 g), Solution 3 (4.0 g), Solution 4 (2.0 g), citric acid (0.4 g) and erythritol (7.6 g) were added and stirred until dissolved. Xanthan gum (1.2 g) was slowly added to the solution while homogenizing. The homogenization speed was slowly increased from low to medium speed as the formulation thickened. Following addition of Xanthan gum, the formulation was homogenized at medium speed for another 3 min. The Xanthan gum appeared fully dispersed (lumps no longer visible). Finally, 20 g of MSP were added to the formulation and homogenized at medium speed for 3 min.

For the preparation of samples for the consumer trial (Example 3), the manufacture of Formulas 4, 12, 13, 14 and 16 was repeated at larger scale (200 g).

TABLE 1
Formulations

| | Ingredient concentration (wt %) | | | | | | |
|---|---|---|---|---|---|---|---|
| | | Citric | Potassium | | | | |
| Formula | MSP | acid | sorbate | Water | Thickener * | Sweetener^{#} | Flavouring ^{¤} |
| Formula 1 | 10 | 0.2 | 0.1 | 88.7 | 1 | — | — |
| Formula 2 | 10 | 0.2 | 0.1 | 87.7 | 2 | — | — |
| Formula 3 | 10 | 0.2 | 0.1 | 88.7 | 1 | — | — |
| Formula 4 | 10 | 0.2 | 0.1 | 87.7 | 2 | — | — |
| Formula 5 | 10 | 0.2 | 0.1 | 88.7 | 1 | — | — |
| Formula 6 | 10 | 0.2 | 0.1 | 87.7 | 2 | — | — |
| Formula 7 | 10 | 0.2 | 0.1 | 88.7 | 1 | — | — |
| Formula 8 | 10 | 0.2 | 0.1 | 87.7 | 2 | — | — |
| Formula 9 | 10 | 0.2 | 0.1 | 88.7 | 1 | — | — |
| Formula 10 | 10 | 0.2 | 0.1 | 87.7 | 2 | — | — |
| Formula 11 | 10 | 0.2 | 0.1 | 88.7 | 1 | — | — |
| Formula 12 | 10 | 0.2 | 0.1 | 87.7 | 2 | — | — |
| Formula 13 | 10 | 0.2 | 0.1 | 89.4 | 0.3 | — | — |
| Formula 14 | 10 | 0.2 | 0.1 | 89.1 | 0.6 | — | — |
| Formula 15 | 10 | 0.2 | 0.1 | 88.7 | 1 | — | — |
| Formula 16 | 10 | 0.2 | 0.1 | 85.04 | 0.6 | 3.81 | 0.25 |

* Guar gum in Formula 1-2, tragacanth gum in Formula 3-4, karaya gum in Formula 5-6, 2-hydroxyethylcellulose in Formula 7-8, sodium carboxymethylcellulose in formula 9-10, colloidal crystalline cellulose in Formula 11-12 and xanthan gum in Formula 13-16
^{#}Erythritol (3.80 wt %) and steviol glycosides (0.01 wt %)
^{¤} Vanilla (0.16 wt %), blueberry (0.06 wt %) and peach (0.03 wt %) flavourings

### Example 2: Evaluation of Suspension Stability

The day following manufacture, approximately 20 mL of each formula was transferred to a glass vial and stored in the dark at room temperature. After six weeks, the samples were observed visually. If separation was observed (clear liquid on top), the suspension was not stable. If no separation was observed after six weeks, the suspension was considered stable under the conditions of this experiment. Results are presented in Table 2.

TABLE 2
Results of suspension stability evaluation

| | | Suspension |
|---|---|---|
| Formula | Thickener | stability |
| Formula 1 | Guar gum 1.0 wt % | Not stable |
| Formula 2* | Guar gum 2.0 wt % | Stable |
| Formula 3 | Tragacanth gum 1.0 wt % | Not stable |
| Formula 4 | Tragacanth gum 2.0 wt % | Stable |
| Formula 5 | Karaya gum 1.0 wt % | Not stable |
| Formula 6 | Karaya gum 2.0 wt % | Not stable |
| Formula 7 | 2-Hydroxyethylcellulose 1.0 wt % | Not stable |
| Formula 8 | 2-Hydroxyethylcellulose 1.0 wt % | Not stable |
| Formula 9 | Sodium carboxymethylcellulose 1.0 | Not stable |
| | wt % | |
| Formula 10 | Sodium carboxymethylcellulose 2.0 | Not stable |
| | wt % | |
| Formula 11 | Colloidal microcrystalline cellulose 1.0 | Not stable |
| | wt % | |
| Formula 12 | Colloidal microcrystalline cellulose 2.0 | Stable |
| | wt % | |
| Formula 13 | Xanthan gum 0.3% | Stable |
| Formula 14 | Xanthan gum 0.6% | Stable |
| Formula 15 | Xanthan gum 1.0% | Stable |
| Formula 16 | Xanthan gum 0.6% | Stable |

*This formula formed a very thick gel that is difficult to pack

### Example 3: Evaluation of Suspension Stability after 1 Year

Some of the samples that appeared stable after six weeks of storage in the dark at room temperature (Example 2) were kept in the same conditions. After 1 year, the samples were observed visually again. If separation was observed (clear liquid on top), the suspension was not stable. If no separation was observed after 1 year, the suspension was considered stable under the conditions of this experiment. Results are presented in Table 3.

TABLE 3
Results of suspension stability evaluation
after 1 year of storage at room temperature

| | | Suspension stability |
|---|---|---|
| Formula | Thickener | after 1 year |
| Formula 2 | Guar gum 2.0 wt % | Stable |
| Formula 12 | Colloidal microcrystalline | Stable |
| | cellulose 2.0 wt % | |
| Formula 13 | Xanthan gum 0.3% | Not stable |
| Formula 14 | Xanthan gum 0.6% | Stable |
| Formula 15 | Xanthan gum 1.0% | Stable |
| Formula 16 | Xanthan gum 0.6% | Stable |

### Example 4: Consumer Trial

Volunteers were given tubes containing approximately 10 mL of each formula selected for the consumer trial. They were instructed to taste a small amount (one tablespoon) of each product, drinking water (at least one gulp) between each product. For each product, they were asked to rate their agreement with the statements listed in Table 4 using a 5 point grading scale. 1 was the lowest and 5 was the highest agreement to the statement.

Feedback from 13 volunteers were collected and average scores were calculated. An average score below 4.0 was considered a fail and an average score of 4.0 or above was considered as a pass. Results are summarised in Table 4.

TABLE 4
Results of consumer trial

| | Formula | Formula | Formula | Formula | Formula |
|---|---|---|---|---|---|
| Statement | 4 | 12 | 13 | 14 | 16 |
| The product has a | Fail | Fail | Fail | Fail | Pass |
| pleasant taste | | | | | |
| The product does not feel | Pass | Fail | Fail | Pass | Pass |
| gritty | | | | | |
| There is no dry mouth | Fail | Fail | Fail | Pass | Pass |
| feeling after taking the | | | | | |
| product | | | | | |
| The product does not feel | Fail | Fail | Pass | Pass | Pass |
| too thick | | | | | |
| I can imagine taking the | Fail | Fail | Fail | Fail | Pass |
| product during each meal | | | | | |

1. An oral dietary supplement comprising:
(a) water;
(b) silica particles; and
(c) a thickening agent,
wherein the thickening agent is Xanthan gum.

2. The dietary supplement of claim 1, wherein the dietary supplement comprises from about 70.0 to about 95.0 wt % water.

3. The dietary supplement of claim 1 or claim 2, wherein the dietary supplement comprises from about 5.0 to about 15.0 wt % of the silica particles.

4. The dietary supplement of any one of claims 1 to 3, wherein the dietary supplement comprises from about 0.4 wt % to about 2.0 wt % of the thickening agent.

5. The dietary supplement of any one of claims 1 to 4, wherein the dietary supplement comprises from about 0.4 wt % to about 1.2 wt % of the thickening agent.

6. The formulation of any one of claims 1 to 5, wherein the dietary supplement further comprises:
(d) one or more preservative, sweetener, flavouring, fragrance and/or colorant.

7. The formulation of any one of claims 1 to 6, wherein the silica particles are porous.
